(19) United States
(12) Patent Application Publication
(10) Pub. No.: US 2026/0042089 A1
(43) Pub. Date: Feb. 12, 2026

(54) METHOD FOR PRODUCING CATION EXCHANGE RESIN AND METHOD FOR REFINING ORGANIC ACID SOLUTION

(71) Applicant: ORGANO CORPORATION, Tokyo (JP)

(72) Inventor: Noriko TAKADA, Tokyo (JP)

(73) Assignee: ORGANO CORPORATION, Tokyo (JP)

(21) Appl. No.: 19/118,492

(22) PCT Filed: Aug. 17, 2023
(86) PCT No.: PCT/JP2023/029696

§ 371 Date: Apr. 4, 2025

(30) Foreign Application Priority Data
Oct. 6, 2022 (JP) 2022-161732

(51) Int. Cl. B01J 49/06; B01D 15/20; B01D 15/36; B01J 39/05; B01J 49/53; C07C 51/47

(52) U.S. Cl. CPC B01J 49/06; B01D 15/203; B01D 15/362; B01J 39/05; B01J 49/53; C07C 51/47

(57) ABSTRACT

Provided is a method for producing a cation exchange resin. The method includes: bringing a mineral acid solution having a metal impurity content of 1 mg/L or less and a concentration of 5 mass % or more into contact with a cation exchange resin (A) to obtain a cation exchange resin (B); bringing an organic acid solution having a metal impurity content of 2 mg/L or less and a concentration of 3 mass % or more into contact with the cation exchange resin (B) to purify the organic acid solution; and bringing a mineral acid solution having a metal impurity content of 1 mg/L or less and a concentration of 5 mass % or more into contact with the cation exchange resin (B) after bringing the organic acid solution into contact with the cation exchange resin (B) to obtain a cation exchange resin (C).

### TECHNICAL FIELD

The present invention relates to a method for producing a cation exchange resin and a method for highly purifying an organic acid solution using the cation exchange resin.

### BACKGROUND ART

In the semiconductor manufacturing process, various high-purity chemical solutions are used, and there is a strong need to reduce metal impurities in various chemical solutions, not limited to ultrapure water. Ion exchange resins (cation exchange resins, anion exchange resins, and chelating resins) are used to remove metal impurities in various chemical solutions. A method is consequently sought for eliminating eluted substances from the ion exchange resins themselves used in the purification of various chemical solutions.

As a method for reducing the metal impurity content of an ion exchange resin, a method has been proposed (Patent Literature 1) in which a high-purity mineral acid solution having a metal impurity content of 1 mg/L or less is brought into contact with an ion exchange resin. A method has also been proposed (Patent Literature 2) for purifying an ion exchange resin using a mineral acid solution containing a chelating agent such as ethylenediaminetetraacetic acid.

### CITATION LIST

### Patent Literature

[PTL 1] JP 2007-117781 A
[PTL 2] JP 2010-234339 A

### SUMMARY OF INVENTION

### Technical Problem

In particular, when ion exchange resins are used to purify organic acid solutions, the amount of metal eluted from the ion exchange resins tends to increase due to the metal dissolution and extraction effects of the organic acid solution itself, which is the purification target (the liquid being treated). Therefore, even when an organic acid solution was purified using an ion exchange resin purified by the method described in the above patent literature, there have been cases in which trace amounts of metal elution became a problem. In addition, in the purification of an organic acid solution, the problem occurs that the life of the used ion exchange resin is short, thus increasing the frequency of replacement of the ion exchange resin, resulting in increased costs.

Therefore, one purpose of the present invention is to provide a method for producing a cation exchange resin in which the metal impurity content of the cation exchange resin is reduced and metal elution is low even when the cation exchange resin is used in purifying an organic acid solution. Another purpose of the present invention is to provide a method for highly purifying an organic acid solution using the cation exchange resin that can be regenerated and utilized.

### Solution to Problem

The present invention is a method for producing a cation exchange resin, the method including: a first regeneration step of bringing a mineral acid solution having a metal impurity content of 1 mg/L or less and a concentration of 5 mass % or more into contact with a cation exchange resin (A) to obtain a cation exchange resin (B); a purification step of bringing an organic acid solution having a metal impurity content of 2 mg/L or less and a concentration of 3 mass % or more into contact with the cation exchange resin (B) to purify the organic acid solution; and a second regeneration step of bringing a mineral acid solution having a metal impurity content of 1 mg/L or less and a concentration of 5 mass % or more into contact with the cation exchange resin (B) after the purification step to obtain a cation exchange resin (C).

In addition, the present invention is a method for purifying an organic acid solution, the method including: a first regeneration step of bringing a mineral acid solution having a metal impurity content of 1 mg/L or less and a concentration of 5 mass % or more into contact with a cation exchange resin (A) to obtain a cation exchange resin (B); and a purification step of bringing an organic acid solution having a metal impurity content of 2 mg/L or less and a concentration of 3 mass % or more into contact with the cation exchange resin (B) to purify the organic acid solution, wherein the cation exchange resin (B) after the purification step is reused as the cation exchange resin (A) used in the first regeneration step, and wherein the first regeneration step and the purification step are further repeated one or more times.

### Advantageous Effects of Invention

According to the present invention, a cation exchange resin can be obtained in which the metal impurity content of the cation exchange resin is reduced and metal elution is low even when the cation exchange resin has been used in purifying an organic acid solution. Moreover, by using the cation exchange resin, the metal impurity content of an organic acid solution can be stably reduced. Furthermore, according to the present invention, a method can be provided for highly purifying an organic acid solution that can suppress increase in cost by regenerating and utilizing the cation exchange resin.

### DESCRIPTION OF EMBODIMENTS

### Method for Producing a Cation Exchange Resin

A method for producing a cation exchange resin with reduced metal elution according to the present invention has the following steps: a first regeneration step of bringing a mineral acid solution having a metal impurity content of 1 mg/L or less and a concentration of 5 mass % or more into contact with a cation exchange resin (A) to obtain a cation exchange resin (B); a purification step of bringing an organic acid solution having a metal impurity content of 2 mg/L or less and a concentration of 3 mass % or more into contact with the cation exchange resin (B) to purify the organic acid solution; and a second regeneration step of bringing a mineral acid solution having a metal impurity content of 1 mg/L or less and a concentration of 5 mass % or more into contact with the cation exchange resin (B) after the purification step to obtain a cation exchange resin (C). Each step is described in detail below.

### First Regeneration Step

In the first regeneration step, a mineral acid solution having a metal impurity content of 1 mg/L or less and a concentration of 5 mass % or more is brought into contact with a cation exchange resin (A) as a regenerant to obtain an H-form (hydrogen ion form) cation exchange resin (B). Here, “a cation exchange resin (A)” means the cation exchange resin before purification, i.e., before regeneration, and “a cation exchange resin (B)” means the cation exchange resin after purification, i.e., after regeneration. This is a step to reduce the amount of metal impurities contained in the cation exchange resin used for purifying an organic acid solution prior to the purification of the organic acid solution.

### Cation Exchange Resin (A)

Any salt-form such as Na-form and K-form or an H-form cation exchange resin can be used as a cation exchange resin (A). However, from the viewpoint of suppressing increase in cost due to an increase of the amount of regenerant required, an H-form cation exchange resin is preferably used as the cation exchange resin (A). The cation exchange resin (A) is not limited, but an organic polymer-based cation exchange resin with an organic polymer matrix is preferred. Styrene-based resins and acrylic-based resins are examples of organic polymers that can be used as a matrix.

In the present description, “styrene-based resin” means a resin containing 50 mass % or more of structural units derived from styrene or a styrene derivative and is obtained by homopolymerizing or copolymerizing styrene or a styrene derivative. Examples of the styrene derivative include α-methylstyrene, vinyltoluene, chlorostyrene, ethylstyrene, i-propylstyrene, dimethylstyrene, and bromostyrene. The styrene-based resin may be a copolymer with another copolymerizable vinyl monomer if the resin contains a homopolymer or a copolymer of styrene or a styrene derivative as the main component. Examples of such a vinyl monomer include one or more selected from divinylbenzenes such as o-divinylbenzene, m-divinylbenzene, and p-divinylbenzene; polyfunctional monomers including alkylene glycol di(meth)acrylate such as ethylene glycol di(meth)acrylate and polyethylene glycol di(meth)acrylate; (meth)acrylonitrile; and methyl(meth)acrylate. Among these, divinylbenzene, ethylene glycol di(meth)acrylate, and polyethylene glycol di(meth)acrylate having 4 to 16 polymerized ethylene units are preferred, divinylbenzene and ethylene glycol di(meth)acrylate are more preferred, and divinylbenzene is especially preferred.

In the present description, “acrylic-based resin” means a resin containing 50 mass % or more of structural units selected from structural units derived from acrylic acid, methacrylic acid, acrylic ester, and methacrylic ester obtained by homopolymerizing or copolymerizing one or more structural units selected from acrylic acid, methacrylic acid, acrylic ester, and methacrylic ester. Examples of the acrylic-based resin include one or more selected from a homopolymer of acrylic acid, a homopolymer of methacrylic acid, a homopolymer of acrylic ester, a homopolymer of methacrylic ester, a copolymer of acrylic acid with other monomers (such as acrylic ester, methacrylic acid, methacrylic ester, α-olefine (such as ethylene and divinylbenzene)), a copolymer of methacrylic acid with other monomers (such as acrylic acid, acrylic ester, methacrylic ester, α-olefin (such as ethylene and divinylbenzene)), a copolymer of acrylic acid ester with other monomers (such as acrylic acid, methacrylic acid, methacrylic ester, α-olefin (such as ethylene and divinylbenzene)), and a copolymer of methacrylic acid ester with other monomers (such as acrylic acid, acrylic ester, methacrylic acid, and α-olefin (such as ethylene and divinylbenzene)). Among these, a methacrylic acid-divinylbenzene copolymer or an acrylic acid-divinylbenzene copolymer is preferred.

As the acrylic acid esters, alkyl esters of acrylic acid are preferred, linear or branched alkyl esters of acrylic acid are more preferred, and linear alkyl esters of acrylic acid are even more preferred. The number of carbon atoms of the alkyl groups included in the alkyl ester moiety is preferably from 1 to 4, and the acrylic acid ester is particularly preferred to be methyl acrylate or ethyl acrylate.

As the methacrylic acid ester, alkyl methacrylate esters are preferred, linear or branched alkyl esters of methacrylic acid are more preferred, and linear alkyl esters of methacrylic acid are even more preferred. The number of carbon atoms of the alkyl group in the alkyl ester moiety is preferably from 1 to 4, and the methacrylic acid alkyl ester is particularly preferred to be methyl methacrylate or ethyl methacrylate.

The matrix of the cation exchange resin (A) can be a transparent gel-type in which the resin has small-diameter pores, a macroreticular (MR)-type, or a microporous-type (also called porous or high porous-type) in which the resin has large-diameter pores.

The cation exchange resin (A) includes a strongly acidic cation exchange resin with sulfonic acid groups and a weakly acidic cation exchange resin with carboxylic acid groups. As the cation exchange resin (A), any of the common resins for producing pure water (e.g., the Amberlite series (product name, manufactured by DuPont de Nemours, Inc.)) can be used. Specifically, examples of the cation exchange resin (A) include but are not limited to: Amberlite (registered trademark) IRN99H (gel-type strongly acidic cation exchange resin), IR120B, IR124, and 200CT (all product names, manufactured by DuPont de Nemours, Inc.); Amberjet (registered trademark) 1060H (gel-type strongly acidic cation exchange resin), 1020, 1024, and 1220 (all product names, manufactured by ORGANO CORPORATION); ORLITE (registered trademark) DS-1 (gel-type strongly acidic cation exchange resin), DS-4 (microporous-type strongly acidic cation exchange resin) (both product names, manufactured by ORGANO CORPORATION); DIAION (registered trademark) SK104H, SK1B, SK110, SK112, PK208, PK212L, PK216, PK218, PK220, PK228, UBK08, UBK10, and UBK12 (all product names, manufactured by Mitsubishi Chemical Corporation); C100, C100E, C120E, C100x10, C100x12, C150, C160, and SGC650 (all product names, manufactured by Purolite K.K.); and MonoPlus S108H, SP112, and S1668 (all product names, manufactured by Lewatit). The cation exchange resin (A) may be used alone or in a combination of two or more.

In addition, as the purified H-form cation exchange resin (B) obtained by bringing a mineral acid solution having a metal impurity content of 1 mg/L or less and a concentration of 5 mass % or more into contact with the cation exchange resin (A), a commercially available cation exchange resin purified by such a method can be used. Specifically, among the cation exchange resins shown in the above examples, the ORLITE series (product name, manufactured by ORGANO CORPORATION) can be used as is as the cation exchange resin (B) in the next step, i.e., the purification step. In other words, when such a cation exchange resin is used, the first regeneration step can be omitted. From the viewpoint of metal removal performance, the cation exchange resin is preferably a strongly acidic cation exchange resin, and more preferably a strongly acidic cation exchange resin having a total exchange capacity of 1.9 eq/L-R or more.

The average particle size of the cation exchange resin is not limited but can be, for example, from 0.1 mm to 1.0 mm. In the present description, the average particle size means a harmonic mean diameter.

In this step, as described in Patent Literature 1, the metal impurities contained in the cation exchange resin (A) are removed and reduced by bringing a mineral acid solution having a metal impurity content of 1 mg/L or less and a concentration of 5 mass % or more into contact with the cation exchange resin (A) to obtain a purified H-form cation exchange resin (B). By bringing a mineral acid solution that contains an extremely low amount of metal impurities into contact with the cation exchange resin (A), the amount of metal impurities contained in the cation exchange resin (A) can be reliably and effectively reduced, and the cation exchange resin (B) with a small amount of eluted metal impurities can be obtained. After the mineral acid solution is brought into contact with the cation exchange resin (A), the resin is preferably washed with pure water or ultrapure water to remove the mineral acid solution remaining in the resin.

The amount of metal impurities contained in the mineral acid solution used as a regenerant is 1 mg/L or less, preferably 0.5 mg/L or less, and more preferably 0.2 mg/L or less. The concentration of the mineral acid solution is 5 mass % or more. If the concentration of the mineral acid solution is less than 5 mass %, a sufficient effect of reducing metal impurities in the cation exchange resin cannot be obtained. The maximum concentration of the mineral acid solution is not limited but is usually 30 mass % or less. The metal impurity contained in the mineral acid solution is a concept that also includes metal impurity ions, representative examples being sodium (Na), magnesium (Mg), calcium (Ca), and iron (Fe). The mineral acid solution is preferably an aqueous solution, and examples include hydrochloric acid, sulfuric acid, and nitric acid. The phrase “bringing a mineral acid solution into contact with the cation exchange resin (A)” includes passing the mineral acid solution through the cation exchange resin (A) as well as immersing the cation exchange resin (A) in the mineral acid solution.

The all-metal impurity content eluted when hydrochloric acid having a concentration of 3 mass % is passed through the cation exchange resin (B) obtained in the first regeneration step at a volume ratio of 25 times is preferably 5 mg/L-R or less, and more preferably 3 mg/L-R or less. The amount of metal impurities contained in the hydrochloric acid having a concentration of 3 mass % is preferably, but not limited to, 1 mg/L or less, more preferably 0.5 mg/L or less, and even more preferably 0.2 mg/L or less for the purpose of improving the accuracy of analysis. The phrase “at a volume ratio of 25 times” of hydrochloric acid means that 25 times by volume of hydrochloric acid is passed through the cation exchange resin (B) relative to the volume of the cation exchange resin (B). The unit “/L-R” means “per liter of volume of the cation exchange resin in a water-wetted condition.” A water-wetted condition refers to a state in which the ion exchanger is immersed in water. The volume of the water-wetted condition can be measured using a measuring instrument such as a measuring cylinder to determine the volume of the ion exchanger immersed in water. The cation exchange resin in a water-wetted condition is obtained by bringing the cation exchange resin into contact with an atmosphere of 100% relative humidity at 25° C. for at least 15 minutes.

The content of sodium (Na), magnesium (Mg), calcium (Ca), and iron (Fe) in the mineral acid solution having a concentration of 5 mass % or more used as the regenerant is preferably 200 μg/L or less for each element. By bringing a mineral acid solution having a low content of these metal impurities into contact with a cation exchange resin, the content of Na, Mg, Ca, and Fe in the cation exchange resin can be reliably and effectively reduced. Similarly, the content of sodium (Na), magnesium (Mg), calcium (Ca), and iron (Fe) in the hydrochloric acid having a concentration of 3% is preferably 200 μg/L or less for each element.

Known methods can be used for bringing the mineral acid solution into contact with the cation exchange resin (A). For example, a method of passing the mineral acid solution through a column or other resin-filled vessel, a pumping unit, or a storage tank can be mentioned. The pumping unit includes a pump employed to circulate the mineral acid solution and a device for expelling compressed air or nitrogen, etc. The storage tank is a container for holding the mineral acid solution or mineral acid solution after treatment. From the viewpoint of safety, the temperature of the mineral acid solution at the time of liquid passage can be, for example, from 15° C. to 60° C. The SV (Space Velocity) of the mineral acid solution that is passed into the resin-filled vessel is not limited, but a low flow rate is preferred as long as operation can be maintained. The SV of the mineral acid solution can be, for example, from 0.5 to 20, preferably 10 or less, and more preferably 5 or less. The SV of the mineral acid solution is preferably lower than the SV of the organic acid solution in the purification step described below. The flow rate multiple of the mineral acid solution to be passed through the resin is preferably from 1 BV to 40 BV relative to the volume of the resin. The above liquid flow conditions are only an example, and each condition can be adjusted as appropriate. The greater the amount of high-purity mineral acid solution used in the regeneration, the greater the amount of the metal impurity content of the resin that can be reduced.

### Purification Step

In the purification step, an organic acid solution is purified by bringing an organic acid solution having a metal impurity content of 2 mg/L or less and a concentration of 3 mass % or more into contact with the cation exchange resin (B).

### Organic Acid Solution

The organic acid solution that is to be purified is not particularly limited as long as it is an organic acid used in the semiconductor manufacturing process. Examples of such organic acids include, but are not limited to, water-soluble organic compounds having a carboxy group such as formic acid, citric acid, oxalic acid, malonic acid, tartaric acid, lactic acid, and malic acid, as well as phosphonic acids. Phosphonic acids are not particularly limited as long as they are organic compounds having a phosphonic acid group (—P═O(OH)₂). Among these, citric acid and oxalic acid are preferred as the organic acid solution.

In the present invention, an organic acid solution having a metal impurity content of 2 mg/L or less and a concentration of 3 mass % or more is used. As the organic acid solution, an organic acid solution having an organic acid concentration of from 3 mass % to 60 mass % is usually used. The total metal impurity content of Na (sodium), Mg (magnesium), Ca (calcium), and Fe (iron) in the organic acid solution before the metal impurity content is reduced (the organic acid solution before purification and that is to be subjected to the purification step) is preferably 2 mg/L or less, and more preferably 1 mg/L or less. An organic acid solution may be used that has been purified by a known method such that the total metal impurity content before purification of the organic acid solution, which is to be subjected to the purification step, falls within the above range. In addition to the impurities mentioned above, the organic acid solution may contain K (potassium), Al (aluminum), Ni (nickel), Cr (chromium), As (arsenic), and any other metal impurities.

A known method can be used as the method for bringing the organic acid solution into contact with the cation exchange resin (B). For example, the same method as the above-described method for bringing the mineral acid solution into contact with the cation exchange resin (A) can be suggested. Specifically, the organic acid solution can be purified by passing the organic acid solution through a column filled with the cation exchange resin (B) (column method). The organic acid solution may be brought into contact with the cation exchange resin (B) by a batch method. The temperature of the organic acid solution at the time of liquid passage can be, for example, from 15° C. to 80° C., taking into consideration the heat resistance temperature of the cation exchange resin as well as the fact that when a solid organic acid is dissolved in an aqueous solution, the organic acid is likely to precipitate at a low temperature. The SV (Space Velocity) of the organic acid solution that is passed into the resin-filled vessel is not limited but is preferably from 1 to 20. The flow rate multiple of the organic acid solution to be passed through the resin is preferably 10 BV or higher relative to the volume of the resin. The upper limit of the flow rate multiple is preferably set by appropriately evaluating in advance the flow rate multiple that satisfies the target. The above liquid flow conditions are only an example, and each condition can be adjusted as appropriate. By performing the above purification step, the amount of each metal impurity in the organic acid solution can be significantly reduced, preferably to 10 μg/L or less, and more preferably to 5 μg/L or less.

### Second Regeneration Step

In the second regeneration step, the cation exchange resin (B) used for purifying the organic acid solution in the purification step described above is again brought into contact with a mineral acid solution having a metal impurity content of 1 mg/L or less and a concentration of 5 mass % or more. This produces a cation exchange resin (C), which is regenerated from the cation exchange resin (B). The second regeneration step is the same as the first regeneration step with the exception that “the cation exchange resin (B)” used for the purification of the organic acid solution, i.e., “the cation exchange resin (B)” after contact with the organic acid solution is used as “the cation exchange resin (A)” in the first regeneration step described above. As shown in the examples, a mineral acid solution having a concentration of 5 mass % or more and a metal impurity content exceeding 1 mg/L used in the second regeneration step was found to result in an increase of the concentration of, in particular, calcium (Ca) in the resin. Therefore, it is important to use a mineral acid solution having a metal impurity content of 1 mg/L or less and a concentration of 5 mass % or more as the regenerant in the first regeneration step and the second regeneration step.

The cation exchange resin (C) is obtained by the first contact of the cation exchange resin with the mineral acid solution, contact with the organic acid solution, and the second contact with the mineral acid solution. Here, the contact between the cation exchange resin and the organic acid solution is a purification step of the organic acid solution in which the metal impurities contained in the organic acid solution are removed by the cation exchange resin. On the other hand, when explained from the perspective of the cation exchange resin, this process can also be described as a purification step of the cation exchange resin in which the impurities that cannot be removed by purification (regeneration) with a mineral acid solution are removed using an organic acid solution. In other words, according to the method of the present invention, while purifying the organic acid solution, which is the liquid being treated, the impurities contained in the cation exchange resin used for the purification that cannot be completely removed by the mineral acid solution can be removed from the cation exchange resin by contact with the organic acid solution, i.e., the liquid being treated. The amount of impurities contained in the cation exchange resin that cannot be completely removed by the mineral acid solution is very small (e.g., at the level of several μg/L or less), and even if this small amount of impurities is eluted into the organic acid solution, these impurities are not expected to affect the advanced purification of the organic acid solution.

The all-metal impurity content eluted when hydrochloric acid having a concentration of 3 mass % is passed through the cation exchange resin (C) obtained in the second regeneration step at a volume ratio of 25 times is preferably 5 mg/L-R or less and more preferably 3 mg/L-R or less, similar to the cation exchange resin (B) obtained in the first regeneration step. Examples of the metal impurities contained in the cation exchange resin include various metals such as sodium (Na), magnesium (Mg), calcium (Ca), iron (Fe), and aluminum (Al). According to the method of the present invention, the content of iron in the cation exchange resin (C) can be lower than the content of iron in the cation exchange resin (B) obtained after the first regeneration step. Specifically, it was found that the content of iron in the cation exchange resin (C) can be reduced to, for example, 70 mass % or less and preferably 50 mass % or less compared to the content of iron in the cation exchange resin (B) obtained after the first regeneration step. Iron is known to form a complex with a mineral acid solution such as hydrochloric acid, and this complex is adsorbed into the cation exchange resin. However, the iron that was adsorbed into the cation exchange resin as a complex was presumably removed from the cation exchange resin due to the chelating action of the organic acid when the organic acid solution was brought into contact with the cation exchange resin. The above effect of reducing the content of iron can be obtained even when the purification step and the second regeneration step are repeated.

The metal impurity content of the cation exchange resin (C) can be further reduced by increasing the amount of regenerant (mineral acid solution) used in the second regeneration step, and this reduction is not limited to iron. Specifically, after the first contact of the cation exchange resin with the mineral acid solution (the first regeneration step), the amount of the mineral acid solution used in the regeneration treatment of the resin after contact with the organic acid solution (the second regeneration step) is set to from 1.0 to 3.0 times the amount used in the first regeneration step. Alternatively, in a case in which the purification step and the second regeneration step are performed repeatedly, after the first contact with the organic acid solution, the mineral acid solution is passed through the cation exchange resin, and the metal concentrations of Ca, Fe, etc. in the mineral acid solution after contact with the cation exchange resin are analyzed to determine, as the required amount of regenerant, the amount of regenerant at which the obtained metal concentrations are reduced to or below a certain level. Then, in the regeneration steps of the second and succeeding steps of contact with the organic acid solution, the regeneration treatment is performed using regenerant in the range of 1.0 to 3.0 times the required amount of regenerant determined above. If the metal removal performance does not satisfy the target value, an operation may be added, at the rate of once every several steps, in which the amount of regenerant is increased or in which the concentration of the regenerant is increased.

### Regeneration and Utilization of Cation Exchange Resin

The life of an ion exchange resin is known to be shorter in the purification of organic acid solutions than in the purification of solutions near neutral (see reference examples described later). In other words, when purifying organic acid solutions, the frequency of replacement of the ion exchange resin increases, leading to increased costs. Therefore, to reduce the substantial frequency of replacement of the ion exchange resin, the regeneration and utilization of the cation exchange resin is preferably performed in the method according to the present invention.

In other words, in the method according to the present invention, the cation exchange resin (C) obtained in the second regeneration step can be reused as the cation exchange resin (B) used in the purification step, and the purification step and the second regeneration step can be repeated one or more times. Thus, according to the present invention, the cation exchange resin (B) used for purification of the organic acid solution after the purification step can be regenerated and used repeatedly for purification of the organic acid solution, whereby the substantial frequency of replacement of ion exchange resin can be reduced. In other words, the cost for purification of organic acid solutions can be reduced.

In the present description, “the cation exchange resin (B) after the purification step” includes both “the cation exchange resin (B) that has undergone the purification step one time” and “when the cation exchange resin (C) obtained by regenerating the cation exchange resin (B) that has undergone the purification step once is used again in the purification step, the cation exchange resin (B) that has undergone the purification step multiple times obtained after the repeated purification step.” Similarly, “the cation exchange resin (C) obtained in the second regeneration step” includes both “the cation exchange resin (C) that has undergone the second regeneration step one time” and “the cation exchange resin (C) that has undergone the second regeneration step multiple times.”

When the cation exchange resin (C) obtained after the second regeneration step is reused as the cation exchange resin (B) in the purification step, the purification step and the second regeneration step can be repeated one or more times, for example, five or more times, 10 or more times, 50 or more times, and 100 or more times. However, when a cation exchange resin is regenerated and used for advanced purification in which the metal concentration in the organic acid solution after purification is at the μg/L level, as in the present invention, the regenerant and the amount of metal impurities in the regenerated resin must be controlled and the deteriorated resin must be replaced as necessary. Organic acids in particular tend to cause swelling and shrinkage of the ion exchange resin. Therefore, the state of deterioration of the resin matrix must be periodically monitored.

Therefore, particularly in a case in which the cation exchange resin (C) obtained after the second regeneration step (which is performed repeatedly) is reused as the cation exchange resin (B) in the purification step, predetermined parameter(s) of the cation exchange resin (C) is (are) preferably measured at predetermined intervals, and part or all of the cation exchange resin (C) that deviates from the predetermined range set in advance for each parameter is preferably accordingly replaced with an H-form cation exchange resin (corresponding to a newly regenerated (not a reused) product, i.e., a new cation exchange resin (B)) that has not been used in the purification of an organic acid solution and that is obtained by being brought into contact with a mineral acid solution having a metal impurity content of 1 mg/L or less and a concentration of 5 mass % or more.

Examples of the predetermined parameters include one or more selected from the group consisting of a metal impurity content of the cation exchange resin, the degree of surface cracking and non-sphericity of the cation exchange resin determined by microscopic observation, and the exchange capacity of the cation exchange resin. Among these, parameters that enable determination of the degree of deterioration of the resin at an early stage being preferred, one or more selected from the group consisting of a metal impurity content of the cation exchange resin, and the degree of surface cracking and non-sphericity of the cation exchange resin as determined by microscopic observation are preferably measured at predetermined intervals to control the degree of deterioration of the resin.

### Metal Impurity Content

When a cation exchange resin is regenerated and used repeatedly for purification of an organic acid solution, a small amount of impurities, etc. that remain during regeneration may conceivably gradually accumulate in the resin. Therefore, the metal impurity content in the resin preferably measured periodically, and if the metal impurity content exceeds a certain amount, some or all of the resin is preferably replaced with a new cation exchange resin. The metal impurity content can be measured, for example, by using hydrochloric acid to elute the metals contained in the resin (hydrochloric acid elution method). Specifically, the all-metal impurity content eluted when hydrochloric acid having a concentration of 3 mass % is passed through the regenerated cation exchange resin (C) at a volume ratio of 25 times is measured using ICP-MS. If there are metals in the resin that cannot be eluted by acid, a microwave method (MW method) in which the resin itself is decomposed to measure the metal content may be used. In addition to analyzing the metal impurity content, the pore distribution and volume of the ion exchange resin, water holding capacity, reaction rate, and exchange capacity as described below may also be analyzed.

The metal impurity content of the regenerated cation exchange resin (C) is preferably low, but a specific guideline for replacement may be, for example, when the metal impurity content exceeds 5 mg/L-R and more preferably when it exceeds 3 mg/L-R. The predetermined range for the above replacement guideline is preferably set appropriately in advance after evaluating the relationship between the metal impurity content in the organic acid solution and the metal impurity content in the resin.

### Degree of Cracking on Resin Surface and Non-Sphericity

Purifying an organic acid solution having a concentration of from 3 mass % to 60 mass % that is used in a semiconductor manufacturing process entails purification of a liquid having a higher density and viscosity than water. In the purification of such an organic acid solution, the pressure tends to increase when the solution is passed through the resin, and the pressure tends to increase further due to the swelling and shrinkage of the resin as well the fragmentation of the resin. Therefore, close attention must be paid not only to changes in the metal removal performance, but also to the effects on the operating conditions, mainly pressure changes during the passage of liquid. The method for measuring the degree of cracking on the surface of the cation exchange resin by microscopic observation includes a method of using a microscope to visually or automatically count the percentage (%) of the resin that has no cracks, chips, or fissures on the resin surface among an arbitrary number of cation exchange resins (PBC: Perfect Beads Content). A method for measuring the non-sphericity by microscopic observation includes a method of using a microscope to visually or automatically count the percentage (%) of resins that maintain their spherical shape among an arbitrary number of cation exchange resins (WBC: Whole Beads Content).

Regarding the degree of cracking on the resin surface (PBC), a specific guideline for replacement is when the degree of cracking becomes 70% or less and preferably 80% or less when the PBC of a new, not yet used resin for purification is set at 100%. Regarding the non-sphericity (WBC), the specific guideline for replacement is when the non-sphericity becomes 80% or less and preferably 90% or less. The predetermined range for the above replacement guideline is preferably set appropriately in advance after evaluating the relationship between the metal impurity content in the organic acid solution and the PBC and WBC of the resin. In the present invention, a particulate removal filter is preferably provided at the latter stage of a resin-filled vessel to prevent particulates of the fragmented ion exchange resin from eluting into the liquid being treated, especially when each step is carried out repeatedly.

### Exchange Capacity

When the exchange capacity decreases due to deterioration of the resin, the treatment performance decreases. Therefore, when the exchange capacity of the cation exchange resin falls below a certain level, the resin is preferably replaced with a new cation exchange resin. The exchange capacity can be measured by a titration method or other methods. Regarding the exchange capacity, a specific guideline for replacement is when the exchange capacity becomes 80% or less and preferably 90% or less than that of unused (new) resin. The predetermined range for the above replacement guideline is preferably set appropriately in advance after evaluating the relationship between the metal impurity content in the organic acid solution and the exchange capacity of the resin.

As explained above, examples of measuring each parameter of the resin include measuring the all-metal impurity content eluted when hydrochloric acid having a concentration of 3 mass % is passed through the resin after regeneration at a volume ratio of 25 times using ICP-MS and measuring the metal impurity content, the exchange capacity, as well as the degree of surface cracking and non-sphericity of the resin before and after regeneration (or before and after being used for purification). By recording the obtained data as quality control data, the time for replacing the resin before the resin deteriorates can be determined based on this data. When replacing the deteriorated resin with newly regenerated resin (the cation exchange resin (B)) that is not a recycled product, either total or partial replacement is acceptable. However, total replacement is preferred from the viewpoint of ease of quality control.

The timing for measuring each parameter, i.e., the predetermined period, is not particularly limited and can be set as appropriate. For example, the measurement may be performed after every purification step, after every second regeneration step, or every time the purification step and the second regeneration step have been repeated once, twice, or five times. Alternatively, in purifying an organic acid solution using the method according to the present invention, the measurement may be performed every week, and the appropriate timing should be set according to the timing of the actual work. Each parameter may be checked either alone or in combination. Although an excessive amount of mineral acid can be used to regenerated and utilized the resin without checking the parameters, this approach is not preferable from the viewpoint of quality control.

### Method for Storing Resin

In the method according to the present invention, the regenerated cation exchange resin may be used immediately for purifying the organic acid solution or may be first stored and then used for purifying the organic acid solution. The cation exchange resin used for purifying the organic acid solution may be regenerated immediately or may be first stored and then regenerated. For example, if the purification of the organic acid solution and the regeneration of the cation exchange resin are carried out at different plants, the cation exchange resin after purification must be transferred.

Here, if the resin is transferred in an organic acid-soaked state (the state of the cation exchange resin after it has been used for purification), the following problems may occur. Organic acids have a high specific gravity, resulting in high transportation costs. Restrictions may be imposed on the transportation of organic acids having a concentration on the order of %. Due to shaking and vibration caused by transportation, organic acids may come into contact with areas that are not usually exposed to acidic liquid, resulting in metal elution. Since the resin swells in the organic acid-soaked state, the resin may be physically damaged if agitated by vibration or other factors. Organic acids with concentrations on the order of % that leak or remain in the piping may crystallize during drying. In particular, organic acids used in semiconductor manufacturing plants have a concentration of around 20 mass %. Organic acids that are dissolved by heating may precipitate due to a drop in temperature. Even if the resin is in a water-soaked state after being regenerated by mineral acid solution and washed with pure or ultrapure water, a large amount of water may increase transportation costs.

Therefore, when the cation exchange resin that follows the purification step used in purifying an organic acid solution is stored for transportation or the like, or when the purification of the organic acid solution is temporarily halted, the organic acid remaining in the cation exchange resin is preferably replaced by water. In other words, the method for producing a cation exchange resin according to the present invention preferably includes, between the purification step and the second regeneration step, a storage step for replacing the organic acid solution remaining in the cation exchange resin (B) after the purification step with water and then storing the cation exchange resin (B) in which the acid solution has been replaced by water until it is to be used in the second regeneration step. Pure water or ultrapure water can be used as the replacement water. As described above, even when the resin is in a water-soaked state, a large amount of water may result in increased transportation cost, and the resin after replacement by water may therefore be dehydrated by a known method as necessary. The above-mentioned “cation exchange resin after the purification step used in purifying an organic acid solution” may be the cation exchange resin after being used once for the purification of the organic acid solution (the cation exchange resin (B) after the first purification step) or may be the cation exchange resin (B) after the purification and regeneration of an organic acid solution have been repeated multiple times.

No particular limitations apply to the liquid passage conditions when replacing with water the organic acid in the cation exchange resin after use for purifying an organic acid solution, but, for example, SV10 or less and about 2BV to 30BV are preferable. At this time, the pH of the aqueous solution at the outlet of the resin tower filled with the cation exchange resin may be checked, and the resin may be washed with water until it exhibits weak acidity to neutrality. Diluting the organic acid concentration in the cation exchange resin to a certain level and reducing to a concentration that does not readily result in precipitation is sufficient.

### Method for Purifying Organic Acid Solution

The method for purifying an organic acid solution according to the present invention is a method for purifying an organic acid solution using the cation exchange resin obtained by the first regeneration step or the second regeneration step in the above method for producing the cation exchange resin. In other words, the method for purifying an organic acid solution according to the present invention has the following steps: a first regeneration step in which a mineral acid solution having a metal impurity content of 1 mg/L or less and a concentration of 5 mass % or more is brought into contact with a cation exchange resin (A) to obtain a cation exchange resin (B); and a purification step in which an organic acid solution having a metal impurity content of 2 mg/L or less and a concentration of 3 mass % or more is brought into contact with the cation exchange resin (B) to purify the organic acid solution. The method for purifying the organic acid solution according to the present invention is characterized in that the cation exchange resin (B) after the purification step is reused as the cation exchange resin (A) used in the first regeneration step, and the first regeneration step and the purification step are repeated one or more times. According to this method, the cation exchange resin used for purification can be regenerated and utilized, thus enabling a reduction of the substantial frequency of replacement of the cation exchange resin.

The first regeneration step and the purification step in the method for purifying the organic acid solution according to the present invention are steps corresponding to the first regeneration step and the purification step, respectively, in the above-described method for producing the cation exchange resin according to the present invention, and a detailed description is therefore here omitted. The second or subsequent first regeneration steps in which the cation exchange resin (B) after the purification step is reused as the cation exchange resin (A) in the first regeneration step can be said to correspond to the second regeneration step in the method for producing the cation exchange resin according to the present invention described above.

The regeneration and utilization of the cation exchange resin and the method for storing the resin described in the above-mentioned method for producing the cation exchange resin according to the present invention can also be appropriately applied to the method for purifying an organic acid solution according to the present invention.

By using the purification method according to the present invention, for example, an organic acid solution can be purified according to the following procedure. As a resin-filled vessel, for example, a container of water purification cartridge size (several hundred mL) to gas cylinder size (several L to 70 L), or column size (50 L to 100 L) is used, and the container is filled with the cation exchange resin (B) obtained in the first regeneration step. Next, the liquid being treated (organic acid solution) is passed through the container to obtain a purified treated liquid (the initial purification step). The cation exchange resin (B) obtained from the purification step is washed with water while still in the resin-filled vessel and then dehydrated. Next, the cation exchange resin (B) obtained by washing with water is reused as the cation exchange resin (A) and regenerated (the next (second-time) first regeneration step). The quality control of the regenerated resin and estimation of the replacement time of the resin is performed based on the results of the quality control of the regenerated resin and the evaluation of the elution from the regenerated resin (conductivity, resistivity, and TOC). The regenerated cation exchange resin (B) is then used (if necessary, a part or all of the cation exchange resin is replaced) to purify the organic acid solution again (the second (second-time) purification step). The above steps are repeated as appropriate.

### Application of Anion Exchange Resin

In the above description, we have described a method for producing a cation exchange resin and a method for purifying an organic acid solution using the cation exchange resin, but the present invention can also be applied when an anion exchange resin is used in place of the cation exchange resin. In an embodiment using an anion exchange resin, for example, the ORLITE series (product name, manufactured by ORGANO CORPORATION) can be used as the regenerated anion exchange resin after the first regeneration step, but the anion exchange resin employed is not limited thereto.

### EXAMPLES

### Reference Example 1: Purification of an Organic Acid Solution Using High-Purity Purification Resin

Using an H-form strongly acidic cation exchange resin (product name: ORLITE DS-1, manufactured by ORGANO CORPORATION) that has been highly purified by means of the first regeneration step according to the present invention, 30 mass % citric acid (Wako special grade, manufactured by FUJIFILM Wako Pure Chemical Corporation, diluted by ultrapure water) was purified by passage through the resin at SV5 for 6 hours (total 30 BV). The metal concentrations in the citric acid before and after purification were analyzed using Agilent 8900 triple quadrupole ICP-MS (product name, Agilent Technologies Japan, Ltd.). The results are shown in Table 1.

As shown in Table 1, the metal concentrations in 30 mass % citric acid before purification were as high as approximately 800 μg/L, but all of these concentrations could be reduced to 5 μg/L or less. In other words, metal impurities could be significantly reduced from the sub-mg/L order to a single-digit μg/L level.

### Reference Examples 2 and 3: Confirmation of Resin Life in Organic Acid Purification

Using an H-form strongly acidic cation exchange resin (product name: ORLITE DS-1, manufactured by ORGANO CORPORATION) that has been highly purified by means of the first regeneration step according to the present invention, 30 mass % citric acid (Wako special grade, manufactured by FUJIFILM Wako Pure Chemical Corporation, diluted by ultrapure water) was purified by passage through the resin at SV5 for 12 hours (total 60 BV). The metal concentrations in the citric acid before and after purification were analyzed in the same manner as in Reference Example 1 (Reference Example 2).

In addition, to demonstrate that the resin life in the purification of a strongly acidic organic acid solution is short, a simulation solution containing metal impurities equivalent to or greater than the 30 mass % citric acid was purified under the same conditions (at SV5 for 12 hours, total 60 BV) using the same H-form strongly acidic cation exchange resin. The metal concentrations in the simulation solution before and after purification were analyzed in the same manner as in Reference Example 1 (Reference Example 3). The simulation solution was prepared by adding a mixed standard solution for ICP-MS (product name: XSTC series, manufactured by SPEX CertiPrep) to pure water to dilute the solution with pure water.

The results of the purification tests of Reference Examples 2 and 3 are shown in Table 1, along with the results of the purification test of Reference Example 1.

TABLE 1

| | Metal concentrations (μg/L) | | | | |
|---|---|---|---|---|---|
| Metal | Citric acid | | | Simulation solution | |
| elements | | Reference | Reference | Before | Reference |
| contained/ | Before | Example 1 | Example 2 | purifi- | Example 3 |
| pH | purification | 30BV | 60BV | cation | 60BV |
| Na | 789 | <5 | 270 | 820 | <5 |
| Mg | 38 | <5 | <5 | 750 | <5 |
| Al | 7 | <5 | <5 | 541 | <5 |
| K | 139 | <5 | <5 | 734 | <5 |
| Ca | 190 | <5 | <5 | 572 | <5 |
| Cr | 6 | <5 | <5 | 449 | <5 |
| Fe | 8 | <5 | <5 | 610 | <5 |
| As | 7 | <5 | 7 | 404 | <5 |
| pH | <1.0 | <1.0 | <1.0 | 5.1 | 5.0 |

As shown in Table 1, the simulation solution before purification had a higher concentration of metal impurities than the 30 mass % citric acid solution before purification, but no metal elements exceeding 5 μg/L were observed after passage at 60 BV (Reference Example 3). On the other hand, in the purification test of 30 mass % citric acid, 270 μg/L of Na was eluted after passage at 60 BV (Reference Example 2). In other words, at the time of passage at 60 BV, Na in the citric acid remained in the citric acid without being adsorbed by the resin. In addition, although the concentration of As in the stock solution is lower than that of Na, no elution was observed after passage at 30 BV, whereas after passage at 60 BV, elution reached a concentration equivalent to that of the stock solution. These results confirm that the metal elution occurs earlier in the purification of organic acid solutions than in the purification of pure water or near-neutral solutions and that the frequency of resin regeneration is higher.

### Example 1

### First Regeneration Step

An H-form strongly acidic cation exchange resin (product name: ORLITE DS-1, manufactured by ORGANO CORPORATION, cation exchange resin (B)), which had undergone the first regeneration step according to the present invention using a mineral acid solution having a metal impurity content of 1 mg/L or less and a concentration of 5 mass % or more was prepared. The all-metal impurity content eluted when hydrochloric acid having a concentration of 3 mass % was passed through the cation exchange resin (B) at a volume ratio of 25 times was analyzed using ICP-MS (hydrochloric acid elution 1). The content was 5 mg/L-R or less.

### Purification Step

A column (diameter: 19 mm, height: 300 mm) was filled with the cation exchange resin (B), and a 30 mass % citric acid simulation solution (having a metal impurity content of 2 mg/L or less, and a total metal impurity content of Na, Mg, Ca, and Fe of 2 mg/L or less) was passed through the column at SV5. The 30 mass % citric acid simulation solution was prepared by dissolving Wako special grade citric acid (manufactured by FUJIFILM Wako Pure Chemical Corporation) in pure water to a concentration of 30 mass % and then adding a mixed standard solution for ICP-MS (XSTC series, manufactured by SPEX CertiPrep). Ultrapure water was then passed through the resin at SV10 for at least 2 hours to confirm that the pH of the wash water was weakly acidic (pH 4) and that the citric acid concentration was sufficiently low.

### Second Regeneration Step

As a regeneration treatment, the second regeneration step according to the present invention was carried out by bringing a mineral acid solution having a metal impurity content of 1 mg/L or less and a concentration of 5 mass % or more into contact with the cation exchange resin (B) that had been used in the purification step (the mineral acid solution passing through at a lower flow rate than that of the citric acid simulation solution in the purification step). The all-metal impurity content eluted when hydrochloric acid having a concentration of 3 mass % was passed through the obtained cation exchange resin (C) at a volume ratio of 25 times was analyzed using ICP-MS (hydrochloric acid elution 2) and was found to be 5 mg/L-R or less. The ratio of each of the metal concentrations obtained in hydrochloric acid elution 1 before passage of organic acid and in hydrochloric acid elution 2 after passage of organic acid (metal concentration after passage of organic acid/metal concentration before passage of organic acid) was then calculated. The results are shown in Table 2.

TABLE 2

| | Example 1 |
|---|---|
| | Ratio of metal concentrations |
| Metal | before and after pasage of |
| elements | liquid (after pasage of liquid/ |
| contained | before pasage of liquid) |
| Na | <1 |
| Mg | 1.5 |
| Al | <1 |
| Ca | <1 |
| Fe | <0.5 |

As shown in Table 2, the cation exchange resin (C) obtained after further regeneration with a mineral acid after passage of the organic acid showed a tendency to have, in particular, markedly lower Fe content compared to the cation exchange resin (B) obtained in the first regeneration step. The passage of organic acid is believed to reduce the Fe contained in the ion exchange resin by the chelating action of the organic acid at the same time as the ion exchange reaction carried out to remove metals in the organic acid. Based on these points, bringing the cation exchange resin in contact with a mineral acid, an organic acid, and a mineral acid in that order was found to enable reduction of both metals contained in the cation exchange resin that are difficult to remove only with a mineral acid as well as metals that can be removed with a mineral acid and thus enable a highly purified cation exchange resin to be obtained.

### Example 2

A column (diameter: 19 mm, height: 300 mm) was filled with, as the cation exchange resin (B) that was subjected to the first regeneration step (the initial regeneration step) according to the present invention, i.e., the same H-form strongly acidic cation exchange resin as in Example 1. The 30 mass % citric acid simulation solution used in Example 1 (having a metal impurity content of 2 mg/L or less, and a total metal impurity content of Na, Mg, Ca, and Fe of 2 mg/L or less) was passed through the column (flow rate of SV3), followed by washing with ultrapure water in the same manner as in Example 1 (initial purification step). Thereafter, the second regeneration step according to the present invention was carried out on the cation exchange resin (B) used in the purification (passing through at a flow rate lower than that of the citric acid simulation solution in the purification step) using an EL grade mineral acid solution (having a metal impurity content of 1 mg/L or less and a concentration of 5 mass % or more), and then washed with ultrapure water (second-time regeneration step). In this way, the mineral acid regeneration (regeneration step) and the passage of organic acid (purification step) were carried out a total of four times to purify the organic acid solution. The metal concentrations in the organic acid solution before and after the first to fourth purifications of each organic acid solution was measured using Agilent 8900 triple quadrupole ICP-MS (product name, Agilent Technologies Japan, Ltd.) to evaluate the metal removal performance. The metal removal performance was evaluated by calculating the metal removal rate for each metal based on the formula shown below. The results are shown in Table 3.

Metal removal rate (%)={(metal concentrations before purification−metal concentration after purification)/(metal concentration before purification)}×100

After the fourth purification of the organic acid solution, a regeneration process was further performed, and the metal impurity content eluted when hydrochloric acid having a concentration of 3 mass % was passed through the cation exchange resin after the regeneration treatment at a volume ratio of 25 times was analyzed. The measurement was performed using Agilent 8900 triple quadrupole ICP-MS (product name, Agilent Technologies Japan, Ltd.). The metal impurity content thus obtained was compared with that of a new cation exchange resin (resin after the regeneration step) that had not been used in the purification of an organic acid solution. For each metal, the ratio of the metal impurity content of the resin after fourth purification/the metal impurity content of the new resin is shown in Table 4.

TABLE 3

Metal removal rate from organic acid solutions (%)

| | 1st | 2nd | 3rd | 4th |
|---|---|---|---|---|
| Na | 100 | 100 | 100 | 100 |
| Mg | 100 | 100 | 100 | 100 |
| Al | 100 | 99 | 100 | 99 |
| K | 100 | 99 | 99 | 99 |
| Ca | 100 | 100 | 100 | 100 |
| Fe | 100 | 100 | 100 | 100 |
| Ni | 99 | 93 | 98 | 99 |

TABLE 4

| | Ratio of metal impurity | All-metal impurity content | |
|---|---|---|---|
| | content (resin after fourth | | Resin after |
| | purification/new resin) | New resin | fourth purification |
| Na | 1.5 | 1 mg/L-R or less | 1 mg/L-R or less |
| Mg | 1.0 | | |
| Al | 1.5 | | |
| Ca | 2.8 | | |
| Fe | 0.65 | | |

As shown in Table 3, the metal removal rate from the organic acid solution exceeded 90% even when the organic acid solution was purified using a resin that had been used repeatedly for the purification of the organic acid solution. In particular, for metals other than Ni, a consistent metal removal rate of 99% or more was observed even in the fourth purification. Thus, according to the present invention, it was confirmed that even resin that had been used repeatedly for the purification of the organic acid solution can be regenerated and utilized by regenerating the resin with a mineral acid solution.

The results in Table 4 show that metals such as Ca increase with repeated use, but that Fe tends to decrease with repeated purification and regeneration. Furthermore, the all-metal impurity content of the cation exchange resin was 5 mg/L-R or less even when purification and regeneration were repeated, indicating that the resin can be further reused to purify organic acid solutions.

### Comparative Example 1

A column (diameter: 19 mm, height: 300 mm) was filled with cation exchange resin resulting from purifying a citric acid simulation solution in the same manner as in Example 1. The second regeneration step was then carried out by passing a mineral acid solution having a metal impurity content exceeding 1 mg/L through the resin at a flow rate lower than that of the citric acid simulation solution. The metal impurity content of the cation exchange resin (C) obtained after this regeneration was analyzed in the same manner as in Example 1.

As a result, the amount of Ca contained in the cation exchange resin (C) after regeneration was five times or more the amount of Ca contained in the cation exchange resin (C) after the second regeneration step in Example 1.

### Example 3

20 mass % citric acid (Wako special grade, manufactured by FUJIFILM Wako Pure Chemical Corporation, diluted by ultrapure water, having a metal impurity content of 2 mg/L or less, and a total metal impurity content of Na, Mg, Ca, and Fe of 2 mg/L or less) was poured into a 250-ml beaker. The same cation exchange resin as in Example 1 was immersed in the acid solution as the cation exchange resin (B) that was subjected to the first regeneration step according to the present invention, and allowed to stand for 30 minutes (purification step). 5 BV of 20 mass % citric acid was used relative to the cation exchange resin (B). After standing, the citric acid was removed, and the cation exchange resin (B) that had undergone contact with the citric acid was regenerated by immersing the resin in a mineral acid solution having a metal impurity content of 1 mg/L or less and a concentration of 5 mass % or more for 30 minutes (5 BV of the mineral acid solution is used), and then washed with ultrapure water (second regeneration step). This purification step and the second regeneration step were further repeated 10 times, and the PBC, WBC, and exchange capacity of the obtained resin sample were then measured.

The PBC was calculated by using a microscope (product name: Digital Microscope, manufactured by KEYENCE CORPORATION) to visually count the percentage (%) of the cation exchange resin that had no cracks, chips, or fissures on the resin surface. The WBC was calculated by using the above microscope to visually count the percentage (%) of the cation exchange resin that maintained a spherical shape. Furthermore, the exchange capacity was determined by passing an acid through the cation exchange resin prepared in salt-form and determining the number of moles of acid consumed by ion exchange by neutralization titration. The results are shown in Table 5. In the table, the “ultrapure water” condition refers to the condition in which the above cation exchange resin (B) was immersed in the same amount of ultrapure water as the above citric acid, and this state corresponds to a new resin (that has undergone the first regeneration step) that has not yet been used for purification of an organic acid solution. In Table 5, the PBC values for each condition are shown, where 100% is the ultrapure water condition of PBC.

TABLE 5

| | PBC | WBC | Exchange capacity |
|---|---|---|---|
| Conditions | (%) | (%) | (eq/L-R) |
| Ultrapure water | 100 | >99 | >2.0 |
| Citric acid purification + | 100 | >99 | — |
| Regeneration | | | |
| (1 time) | | | |
| Citric acid purification + | 89 | >99 | >2.0 |
| Regeneration | | | |
| (11 times) | | | |

As shown in Table 5, when the purification and regeneration of the organic acid solution was carried out a total of 11 times, no change occurred in the WBC, but a decrease in the PBC was observed. If the PBC is less than 90% relative to a new resin (ultrapure water condition in Table 5), the resin can still be used for repeated purification. From these results, using the PBC rather than the WBC as an index for measuring the deterioration of the resin was found to enable comprehension of damage to the resin matrix at an early stage. Furthermore, when the purification and regeneration of the organic acid solution was carried out a total of 11 times, a decrease in the PBC was observed, but there was no effect on the exchange capacity itself. In other words, repeated purification and regeneration of the organic acid solution was found to have a greater effect on the matrix, such as cracks in the resin, than on the exchange capacity.

The invention includes the following configurations:

### Configuration 1

A method for producing a cation exchange resin, the method comprising:
a first regeneration step of bringing a mineral acid solution having a metal impurity content of 1 mg/L or less and a concentration of 5 mass % or more into contact with a cation exchange resin (A) to obtain a cation exchange resin (B);
a purification step of bringing an organic acid solution having a metal impurity content of 2 mg/L or less and a concentration of 3 mass % or more into contact with the cation exchange resin (B) to purify the organic acid solution; and
a second regeneration step of bringing a mineral acid solution having a metal impurity content of 1 mg/L or less and a concentration of 5 mass % or more into contact with the cation exchange resin (B) after the purification step to obtain a cation exchange resin (C).

### Configuration 2

The method for producing the cation exchange resin according to configuration 1, wherein the cation exchange resin (C) obtained in the second regeneration step is reused as the cation exchange resin (B) used in the purification step, and the purification step and the second regeneration step are further repeated one or more times.

### Configuration 3

The method for producing the cation exchange resin according to configuration 1 or 2, wherein the organic acid solution is selected from a group consisting of formic acid, citric acid, oxalic acid, malonic acid, tartaric acid, lactic acid, malic acid, and phosphonic acids.

### Configuration 4

The method for producing the cation exchange resin according to configuration 3, wherein the concentration of the organic acid solution is from 3 mass % to 60 mass % and a total metal impurity content of Na (sodium), Mg (magnesium), Ca (calcium), and Fe (iron) in the organic acid solution before purification that is provided in the purification step is 2 mg/L or less.

### Configuration 5

The method for producing the cation exchange resin according to any of configurations 1 to 4,
wherein an all-metal impurity content eluted when hydrochloric acid having a concentration of 3 mass % is passed through the cation exchange resin (B) obtained in the first regeneration step at a volume ratio of 25 times is 5 mg/L-R or less, and
wherein an all-metal impurity content eluted when hydrochloric acid having a concentration of 3 mass % is passed through the cation exchange resin (C) obtained in the second regeneration step at a volume ratio of 25 times is 5 mg/L-R or less.

### Configuration 6

The method for producing the cation exchange resin according to any of configurations 1 to 5, wherein one or more parameters selected from a group consisting of a metal impurity content, a degree of surface cracking, and a non-sphericity of the cation exchange resin (C) obtained in the second regeneration step that is to be optionally repeated are measured at predetermined intervals, and the cation exchange resin (C) for which a measured parameter deviates from a predetermined range set in advance for each parameter is replaced with a cation exchange resin that has not been used for the purification of an organic acid solution and that is obtained by being placed in contact with a mineral acid solution having a metal impurity content of 1 mg/L or less and a concentration of 5 mass % or more.

### Configuration 7

The method for producing the cation exchange resin according to any of configurations 1 to 6, the method comprising:
a storage step carried out between the purification step and the second regeneration step of substituting the organic acid solution remaining in the cation exchange resin (B) after the purification step with water and storing the cation exchange resin (B) substituted with water until it is used in the second regeneration step.

### Configuration 8

The method for producing the cation exchange resin according to any of configurations 1 to 7, wherein a content of iron (Fe) in the cation exchange resin (C) obtained in the second regeneration step is less than a content of iron (Fe) in the cation exchange resin (B) obtained in the first regeneration step.

### Configuration 9

A method for purifying an organic acid solution, the method comprising:
a first regeneration step of bringing a mineral acid solution having a metal impurity content of 1 mg/L or less and a concentration of 5 mass % or more into contact with a cation exchange resin (A) to obtain a cation exchange resin (B); and
a purification step of bringing an organic acid solution having a metal impurity content of 2 mg/L or less and a concentration of 3 mass % or more into contact with the cation exchange resin (B) to purify the organic acid solution,
wherein the cation exchange resin (B) after the purification step is reused as the cation exchange resin (A) used in the first regeneration step, and
wherein the first regeneration step and the purification step are further repeated one or more times.

1. A method for producing a cation exchange resin, the method comprising:
bringing a mineral acid solution having a metal impurity content of 1 mg/L or less and a concentration of 5 mass % or more into contact with a cation exchange resin (A) to obtain a cation exchange resin (B);
bringing an organic acid solution having a metal impurity content of 2 mg/L or less and a concentration of 3 mass % or more into contact with the cation exchange resin (B) to purify the organic acid solution; and
bringing a mineral acid solution having a metal impurity content of 1 mg/L or less and a concentration of 5 mass % or more into contact with the cation exchange resin (B) after the bringing of the organic acid solution into contact with the cation exchange resin (B) to obtain a cation exchange resin (C).

2. The method for producing the cation exchange resin according to claim 1, wherein the obtained cation exchange resin (C) is reused as the cation exchange resin (B) used during the bringing of the organic acid solution into contact with the cation exchange resin (B), and the bringing of the organic acid solution into contact with the cation exchange resin (B) and the bringing of the mineral acid solution into contact with the cation exchange resin (B) are further repeated one or more times.

3. The method for producing the cation exchange resin according to claim 1, wherein the organic acid solution is selected from a group consisting of formic acid, citric acid, oxalic acid, malonic acid, tartaric acid, lactic acid, malic acid, and phosphonic acids.

4. The method for producing the cation exchange resin according to claim 3, wherein the concentration of the organic acid solution is from 3 mass % to 60 mass % and a total metal impurity content of Na (sodium), Mg (magnesium), Ca (calcium), and Fe (iron) in the organic acid solution before purification of the organic acid solution is 2 mg/L or less.

5. The method for producing the cation exchange resin according to claim 1,
wherein an all-metal impurity content eluted is 5 mg/L-R or less when hydrochloric acid having a concentration of 3 mass % at a volume ratio of 25 times is passed through the cation exchange resin (B) obtained after contact of the cation exchange resin (B) with the mineral acid solution, and
wherein an all-metal impurity content eluted is 5 mg/L-R or less when hydrochloric acid having a concentration of 3 mass % at a volume ratio of 25 times is passed through the cation exchange resin (C) obtained after contact of the cation exchange resin (B) with the mineral acid solution.

6. The method for producing the cation exchange resin according to claim 2, wherein one or more parameters selected from a group consisting of a metal impurity content, a degree of surface cracking, and a non-sphericity of the cation exchange resin (C) obtained after repeated contact of the cation exchange resin (B) with the mineral acid solution are measured at predetermined intervals, and the cation exchange resin (C) for which a measured parameter deviates from a predetermined range set in advance for each parameter is replaced with a cation exchange resin that has not been used for the purification of an organic acid solution and that is obtained by being placed in contact with a mineral acid solution having a metal impurity content of 1 mg/L or less and a concentration of 5 mass % or more.

7. The method for producing the cation exchange resin according to claim 1, the method comprising:
after purification of the organic acid solution and before the contact of the cation exchange resin (B) with the mineral acid solution, substituting with water the organic acid solution remaining in the cation exchange resin (B) after use of the cation exchange resin (B) for purifying the organic acid solution and storing the cation exchange resin (B) substituted with water until it is used during the contact with the mineral acid solution.

8. The method for producing the cation exchange resin according to claim 1, wherein a content of iron (Fe) in the cation exchange resin (C) obtained after contact of the cation exchange resin (B) with the mineral acid solution is less than a content of iron (Fe) in the cation exchange resin (B) obtained after contact of the cation exchange resin (A) with the mineral acid solution.

9. A method for purifying an organic acid solution, the method comprising:
bringing a mineral acid solution having a metal impurity content of 1 mg/L or less and a concentration of 5 mass % or more into contact with a cation exchange resin (A) to obtain a cation exchange resin (B); and
bringing an organic acid solution having a metal impurity content of 2 mg/L or less and a concentration of 3 mass % or more into contact with the cation exchange resin (B) to purify the organic acid solution,
wherein the cation exchange resin (B) after use of the cation exchange resin (B) for purifying the organic acid solution is reused as the cation exchange resin (A), and
wherein bringing the mineral acid solution into contact with the cation exchange resin (A) and bringing the organic acid solution into contact with the cation exchange resin (B) are further repeated one or more times.
